(19) United States
(12) Patent Application Publication
(10) Pub. No.: US 2021/0407632 A1
(43) Pub. Date: Dec. 30, 2021

(54) SYSTEM AND METHOD FOR DISPLAYING ELECTRONIC HEALTH RECORDS

(71) Applicant: KONINKLIJKE PHILIPS N.V., EINDHOVEN (NL)

(72) Inventors: Eric Thomas CARLSON, New York, NY (US); Oladimeji Feyisetan FARRI, Yorktown Heights, NY (US)

(21) Appl. No.: 16/647,163

(22) PCT Filed: Sep. 6, 2018
(86) PCT No.: PCT/EP2018/073921

§ 371 Date: Mar. 13, 2020

Related U.S. Application Data
(60) Provisional application No. 62/557,845, filed on Sep. 13, 2017.

(51) Int. Cl. G16H 10/60

(52) U.S. Cl. CPC G16H 10/60

(57) ABSTRACT

A system (300) configured to analyze electronic medical records comprises: a user interface (310) configured to receive input from a user and to receive a request for patient information; and a processor (320) comprising: a patient cohort generator (350) configured to: (i) track user input; (ii) identify patient information accessed through the user interface as well as patient parameters associated with the patient; (iii) associate patients into a patient cohort based on the patient parameters; (iv) identify, for the patient cohort, types of information most commonly accessed by the users; and (v) associate the identified types of information with the patient cohort; and a record identifier (370) configured to: (i) associate the patient for whom patient information is requested with a patient cohort; and (ii) identify, based on the patient cohort with whom the patient is associated, the types of information associated with that cohort.

### FIELD

The present disclosure is directed generally to methods and systems for displaying electronic health records.

### BACKGROUND

Electronic medical record display interfaces have limited space for display. However, a patient typically has many associated medical records. Displaying all of the content of medical records associated with the patient leads to information overload, and the display space dedicated to portions of medical records restricts the volume of relevant information that clinicians or other users can quickly and easily consume without extensive manipulation of the data. This information manipulation is prohibitively time-consuming.

One solution for this issue of information overload is to display a portion of a medical record on the screen, such as the first few sentences in the most recent couple of documents, or extracted snippets of these documents. However, this inadvertently produces a blind spot situation in which the significant contents of these documents relevant to the prevailing clinical scenario may not appear in the displayed snippets, or meaningful information in historical records may be hidden while less-meaningful information from more recent records are displayed.

### SUMMARY

There is a continued need for improved display of relevant portions of electronic health records.

The present disclosure is directed to inventive methods and systems for identifying, analyzing, and displaying electronic health records. Various embodiments and implementations herein are directed to a medical record display system that generates a patient cohort with associated commonly-utilized medical records. The system tracks users of the interface and identifies which records are commonly consulted for which patients. Patients with similar parameters are clustered into a patient cohort, and the commonly consulted records for that cohort are identified. When the system receives a query for information about a new patient, the most closely related patient cohort is identified, and then, based on the record types associated with that identified patient cohort, relevant patient medical records are identified. The system can then display the identified relevant patient medical records.

Generally in one aspect, a system for analyzing electronic medical records is provided. The system includes a user interface configured to receive input from a user as the user reviews one or more electronic medical records, and further configured to receive a request for patient information. The system also includes a processor including: a patient cohort generator configured to: (i) track the user input; (ii) identify, based on the user input, patient information accessed through the user interface, and further identify one or more patient parameters associated with the patient; (iii) associate two or more patients into a patient cohort based on the one or more patient parameters; (iv) identify, for the patient cohort, one or more types of information most commonly accessed by the users; and (v) associate the identified one or more types of information with the patient cohort; and a record identifier configured to: (i) associate the patient for whom patient information is requested with a patient cohort; and (ii) identify, based on the patient cohort with whom the patient is associated, the one or more types of information associated with that cohort.

According to an embodiment, the system further includes a patient cohort database configured to store information about one or more generated patient cohorts.

According to an embodiment, the patient cohort generator is further configured to identify a specific user of the user interface among a plurality of users, and further configured to track the user input for the identified specific user. According to an embodiment, the identification of the specific user of the user interface is based at least on part on one or more favorable outcomes for one or more patients.

According to an embodiment, the patient cohort generator is further configured to refine the associated identified one or more types of information using data from an additional medical information source.

According to an embodiment, the patient cohort identification by the record identifier is based at least in part on additional obtained information about the patient.

According to an embodiment, the user interface is further configured to display one or more of the identified types of information associated with the identified patient cohort. According to an embodiment, the user interface is further configured to display a portion of the identified types of information associated with the identified patient cohort.

According to another aspect is a method for analyzing electronic medical records. The method includes the step generating a patient cohort, comprising the steps of: (i) tracking the activity of one or more users of an electronic medical record interface; (ii) identifying, based on an analysis of the tracked activity, patient information accessed by the one or more users through the electronic medical record interface, and further identifying one or more patient parameters associated with the patient; (iii) associating two or more patients into a patient cohort based on the one or more patient parameters; (iv) identifying, for the patient cohort, one or more types of information most commonly accessed by the users; and (v) associating the identified one or more types of information with the patient cohort. The method also includes the steps of: receiving, from a user, a request for information about a patient; associating the patient, based on one or more parameters of the patient, with a patient cohort; and identifying, based on the associated patient cohort, the one or more types of information associated with that cohort.

According to an embodiment, the step of generating a patient cohort further comprises identifying a specific user of the electronic medical record interface for tracking.

According to an embodiment, the identification of the specific user of the user interface is based at least on part on one or more favorable outcomes for one or more patients.

According to an embodiment, the method further includes the step of refining the identified one or more types of information associated with the patient cohort using additional medical information.

According to an embodiment, the method further includes the step of analyzing additional obtained medical information about the patient.

According to an embodiment, the method further includes the step of displaying, on a user interface, the one or more types of information associated with the identified cohort. Displaying the one or more types of information associated with the identified cohort may comprise displaying only an identified portion of the one or more types of information.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects discussed herein. The terms “program” or “computer program” are used herein in a generic sense to refer to any type of computer code (e.g., software or microcode) that can be employed to program one or more processors or controllers.

The term “network” as used herein refers to any interconnection of two or more devices (including controllers or processors) that facilitates the transport of information (e.g. for device control, data storage, data exchange, etc.) between any two or more devices and/or among multiple devices coupled to the network. As should be readily appreciated, various implementations of networks suitable for interconnecting multiple devices may include any of a variety of network topologies and employ any of a variety of communication protocols. Additionally, in various networks according to the present disclosure, any one connection between two devices may represent a dedicated connection between the two systems, or alternatively a non-dedicated connection. In addition to carrying information intended for the two devices, such a non-dedicated connection may carry information not necessarily intended for either of the two devices (e.g., an open network connection). Furthermore, it should be readily appreciated that various networks of devices as discussed herein may employ one or more wireless, wire/cable, and/or fiber optic links to facilitate information transport throughout the network.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Various embodiments present a method and system for intelligently selecting snippets to be displayed on an EMR interface. The method begins by categorizing certain users as “expert users” by identifying those users that exhibit patterns of easy interface navigation (e.g., low number of interactions or short time to access the information that they deem relevant to a patient). As these experts use the system, the method tracks the types of information that are accessed (as typed by applying natural language processing to the accessed information and relating to a clinical ontology) and logs this information along with demographic, vital, diagnosis, etc. classifying information about the associated patient. From here, the most important types of information can be listed and ranked across different patient cohorts.

Thereafter, when any user retrieves a particular patient's record, the method identifies that patient's cohort and retrieves the ranked list of information types. The method then performs NLP across the patient's EMR to identify any entries that match the ontological concepts on the ranked list. Thereafter, snippets from the highest ranking entries can be displayed on the home screen for that patient's EMR. Upon clicking the snippet, the user is presented the full entry from which the snippet is taken.

These and other aspects will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles disclosed herein.

FIG. 1 is a flowchart of a method for displaying electronic health records, in accordance with an embodiment.

FIG. 2 is a flowchart of a method for displaying electronic health records, in accordance with an embodiment.

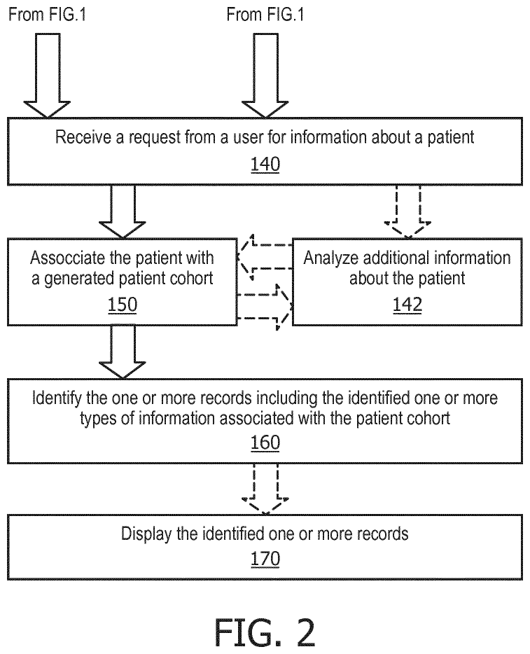

FIG. 3 is a schematic representation of a system for displaying electronic health records, in accordance with an embodiment.

### DETAILED DESCRIPTION OF EMBODIMENTS

The present disclosure describes various embodiments of a system for identifying and displaying electronic health records. More generally, Applicant has recognized and appreciated that it would be beneficial to provide a system that more efficiently utilizes the limited display of a medical record user interface. The system tracks users of the interface and identifies which records are commonly consulted for which patients. Patients with similar parameters are clustered into a patient cohort, and the commonly consulted records for that cohort are identified. When the system receives a query for information about a new patient, the most closely related patient cohort is identified, and then, based on the record types associated with that identified patient cohort, relevant patient medical records are identified. According to an embodiment, the system can display the identified relevant patient medical records on a user interface for review by a clinician, patient, or other user. Since only portions of a medical record can be displayed a user interface at any given time, the system may utilize information from the generated patient cohort and associated records to identify which portion or portions of a record should preferentially be displayed.

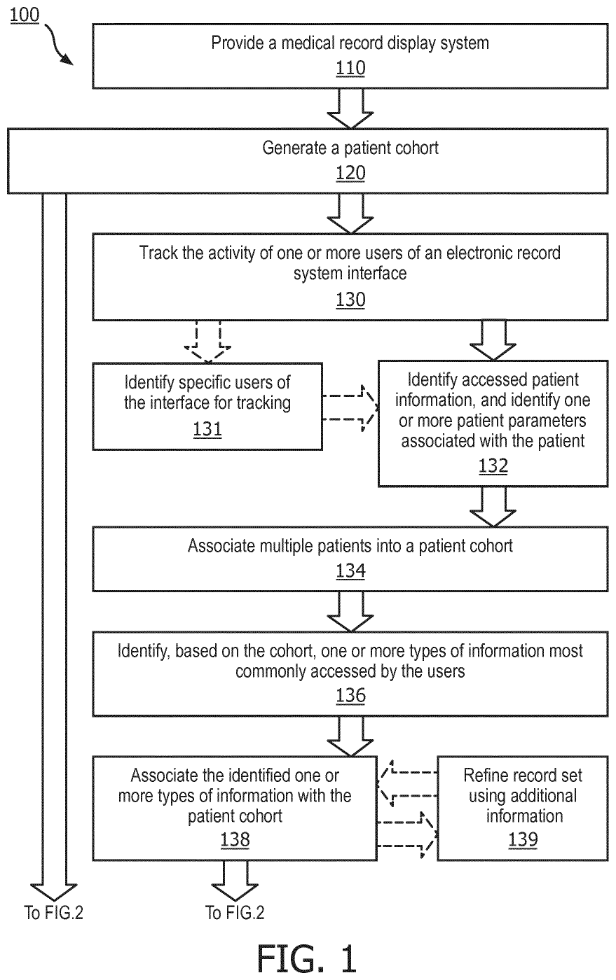

Referring to FIG. 1, in one embodiment, is a flowchart of a method 100 for identifying electronic health records. At step 110 of the method a medical record display system is provided. The medical record display system may be any of the systems described or otherwise envisioned herein.

At step 120 of the method, the medical record system generates a patient cohort. As described below, generating a patient cohort comprises one or more of steps 130 through 138. The patient cohort will comprise a plurality of patients that are related based on one or more parameters. For example, patients may be related based on a clinical context, such as illness, symptoms, treatment, medical history, and/or other clinical contexts. Patients may be related based on patient demographics such as sex, age, background, and/or other patient demographics. The patients may be related based on which record or records a user most commonly accesses or reviews for a patient. Patients may be identified as being related based on a combination of several of these and/or other parameters.

The generated patient cohort will further comprise an identification of one or more types of information, such as medical records, that are most commonly accessed, reviewed, or otherwise utilized by users of the medical record system in regard to the patients in the patient cohort. Thus, if a user of the system frequently accesses X-rays for patients in an orthopedic clinical context, a patient cohort comprising orthopedic patients may have X-rays as one of the types of information associated with that cohort. Accordingly, if a plurality ofpatient cohorts are generated, each cohort may be associated with unique and/or overlapping record types or types of information.

At step 130 of the method, the medical record system tracks the activity of one or more users of the system. For example, the user may be a clinician or other specialist reviewing patient records via a user interface of the system. According to an embodiment, user interface instrumentation tools are utilized to monitor clinician-user interaction with the system. The monitoring aspect of module of the system identifies what patient notes are explored, which records or record types are accessed, and/or other information.

According to an embodiment, at step 131 of the method the system or a user identifies one or more specific users of the system for tracking. These identified specific users will be utilized preferentially over other users, or instead of other users, to identify the patient cohort and/or the record types associated with the patient cohort. For example, a user exhibiting a pattern or history of straightforward navigation or retrieval may be considered an expert user as they will immediately go to or otherwise retrieve relevant textual information. Various metrics may be used in this manner to identify expert users such as, for example, time spent using the interface per session, average time between interface clicks, the number of times the user clicks “back,” the complexity of a “tree” constructed of the user's navigation (e.g., did the user encounter many “dead ends” before finding the desired information or did the user directly access the desired information). In alternate embodiments, a clinician that is determined to make decisions that result in positive outcomes may be identified as the specific user of the system for tracking, which may help refine the expert group. In some embodiments, expert users may be considered experts for all patients for the purposes of identifying most relevant information, or expert status may be conferred on a per-cohort basis. For example, clinician A may be designated an expert for cohort A (e.g., Cardiology patients>40 years old) and thus used for determining most relevant information but may be non-expert for cohort B (e.g., pediatric hematology patients) and thus other experts may be used to identify relevant information for this cohort. One or more specific users may also be identified by a programmer or user of the medical records system. For example, the programmer or user may desire to have a ranking clinician or an experienced user as the identified specific user for analysis and tracking.

According to an embodiment, the system tracks the specific information reviewed or accessed by the user. This specific information can be in addition to or an alternative to tracking the record type accessed by the user. The system may then identify the specific information within a record or a record type commonly accessed by the user. This may be utilized downstream to help identify record types and information within record types to provide to a clinician. According to an embodiment, the system may analyze the specific information accessed by the user to identify similar or related specific information in other records or other record types in order to provide the most relevant information to the clinician.

According to an embodiment, the medical record system utilizes eye-tracking software or algorithms to track or identify information most commonly reviewed or accessed by the users of the medical record system. For example, the user interface or system may comprise or otherwise be in communication with a camera, such as a camera of a wearable device, that identifies and tracks the objects, records, or areas of the user interface that are most commonly and/or most intently reviewed by the user. These objects, records, or areas may be identified as being the most accessed or important objects, records, or areas.

According to an embodiment, the medical record system utilizes natural language processing (NLP) to identify and/or extract information from one or more records identified by the user via tracking. For example, the user may utilize the user interface to access and review unstructured reports or data such as handwritten notes. The medical record system identifies the record accessed or reviewed by the user via tracking such as eye-tracking, and extracts information from that record using NLP or other data extraction or analysis methods.

At step 132 of the method, the system identifies, based on an analysis of the tracked activity, patient information accessed by the one or more users through the electronic medical record interface. For example, the system may log the information or record sources accessed by one or more users and store the information in a database. The system can then retrieve this stored information for immediate or downstream analysis as described or otherwise envisioned herein.

Also at step 132 of the method, the system identifies one or more patient parameters associated with the patient in order to create the patient cohort of related patients. For example, patients may be related based on a clinical context, such as illness, symptoms, treatment, medical history, and/or other clinical contexts. Patients may be related based on patient demographics such as sex, age, background, and/or other patient demographics. The patients may be related based on which record or records a user most commonly accesses or reviews for a patient. Patients may be identified as being related based on a combination of several of these and/or other parameters.

At step 134 of the method, the system associates two or more patients into a patient cohort based on the one or more patient parameters. Patients with similar parameters can be associated into the same patient cohort. Similarity can be based on a threshold, a number of similar or dissimilar parameters, severity or range of one or more of the parameters, input from a programmer or user of the system, demographics, record types, illness, and/or many other patient factors. The patient cohort may be generated or stored in a memory or database, or may otherwise be identified or generated.

At step 136 of the method, the system identifies one or more types of information most commonly accessed by the users for the particular patient cohort. For example, the system may log the information or record sources accessed by one or more users and identify which of the logged sources are utilized most frequently. This could be based on a threshold, a ranking, and/or a machine learning mechanism. Different patient cohorts may have the same commonly accessed record types, some overlapping commonly accessed record types, or non-overlapping commonly accessed record types. In some embodiments the device may utilize the access history of only the expert users (across all cohorts or for this particular cohort) to identify which types of information the expert users most frequently access for patients of this cohort. The method may, at this step, apply natural language processing to extract concepts identified by a clinical ontology from documents accessed by experts for patients in this cohort, and then rank the concepts in terms of frequency of access.

At step 138 of the method, the system associates the identified one or more types of information or records with the patient cohort. The identified commonly accessed record types may be associated with the patient cohort in a memory or database, or may otherwise be identified or associated with the patient cohort. Accordingly, when a clinician or user accesses a patient in the patient cohort, that patient and/or patient cohort will be associated with the identified one or more types of information or records.

At step 139 of the method, the system modifies the patient cohort and/or the identified one or more types of information or records associated with the patient cohort using additional information. For example, one or more patients in the cohort and/or one or more identified records can be ranked, filtered, added, removed, or otherwise modified using clinical databases or other sources of relevant information. As an example, clinical concepts linked to the cohort for diagnostic or therapy decisions can be identified using the additional information and can thus be preferentially reported. Among the many sources of additional information are databases such as Medscape, PubMed, Wikipedia, medical journals, other knowledge-based databases, clinician-curated data, and many more sources.

A plurality of patient cohorts may be generated once or multiple times using a large corpus or database of patients, patient records, and user tracking information. The generated plurality of patient cohorts may be stable, may be updated continuously or periodically, and/or may be newly-formed on demand Once a patient cohort is created, the medical records system utilizes the plurality ofpatient cohorts to optimize the information provided to a clinician for future patients. Accordingly, at step 140 of the method, the medical records system receives a request for information about a patient. The request can be from a clinician or any other user of the medical records system, including a patient. The information about the patient can be medical history, patient parameters, and/or medical records, among many other types of information.

At step 150 of the method, the system associates the patient with one of the generated plurality of patient cohorts, based at least in part on one or more patient parameters. The patient may be associated with a patient cohort for which the patient is most similar. Similarity may be based on a threshold, a number of similar or dissimilar parameters, severity or range of one or more of the parameters, input from a programmer or user of the system, demographics, record types, illness, and/or many other patient factors. The patient's association with a patient cohort may be generated or stored in a memory or database, or may otherwise be identified.

At step 142 of the method, the system analyzes additional information about the patient to facilitate identification of the proper patient cohort, and/or to reduce redundancy in the system. For example, once a new patient is identified in a patient cohort, the patient's notes, social media, activity patterns, and/or other data sources can be analyzed, such as using semantic or natural-language processing. This information may modify or further refine to which patient cohort the patient belongs, or may modify or further refine which records associated with the identified patient cohort are provided or preferred.

At step 160 of the method, the system identifies, based on the patient cohort with which the patient is associated, one or more records of the patient that include information matching the one or more identified types of information associated with the patient cohort. For example, starting with the ranked list of ontological concepts described above with respect to step 136, use NLP to extract any ontological concepts from this patient's records and then compare to the ranked list of concepts for the cohort to determine which documents include concepts that match the list. The system may then select a number of documents (e.g., a preconfigured number or a number that may be displayed on the UI according to the current display configuration and the size of snippets to be displayed as explained below) to be displayed. For example, the system may select the documents matching the highest ranked concepts in the ranked list. In some embodiments, to avoid cumulative information, the system may select only one document for each concept in the ranked list. In such embodiments, for example, even if three documents include concept #1 in the ranked list, the system may select only one (e.g., based on the most recent, the additional concepts included in the document, at random, or based on other selection criteria), and move on to select a document that includes concept #2. Accordingly, this optimizes the information provided to the clinician based on the patient being associated with the proper patient cohort.

According to an embodiment, the system identifies, highlights, or otherwise provides specific portions or snippets of the identified records. These identified portions or snippets may be based on the identified record type, information about the patient, identification of preferred snippets or portions based on user analysis described above, or using any other method.

At step 170 of the method, the identified one or more records are displayed in some form. For example, the interface may display one or more of an identification of the documents (e.g., “radiology report dated 1/1/2017”), a link to the identified document, a snippet of text or image data from the document, or the entire document. The identified information may be presented using any method or system. For example, the information may be presented to the user in real-time, such as via a user interface of a mobile device, laptop, desktop, wearable device, or any other computing device. The results may be presented by any user interface that allows information to be presented, such as a microphone or text input, among many other types of user interfaces. Alternatively, the results may be presented to a computing device or an automated system. In some embodiments, an area of a patient dashboard may be designated for displaying the results of this method. The dashboard may be displayed in response to a selecting or other identification of the patient from another screen of the user interface (e.g., patient search or ward overview) include other information about the patient such as demographic information, vitals, assigned staff, clinical decision support algorithm output, etc.

According to an embodiment, the system preferentially displays identified portions or snippets of the identified records. These identified portions or snippets may be based on the identified record type, information about the patient, identification of preferred snippets or portions based on user analysis described above, or using any other method. In some embodiments, the system may select snippets ofthe text (or image or other data) near the location that the ontological concept matching the ranked list was extracted. According to an embodiment, the portions or snippets are displayed together with links to provide evidence for or additional information about one or more clinical issues in the patient record. According to an embodiment, the portions or snippets are displayed together with links to clinical databases indicating the clinical value of the one or more clinical issues for patient treatment.

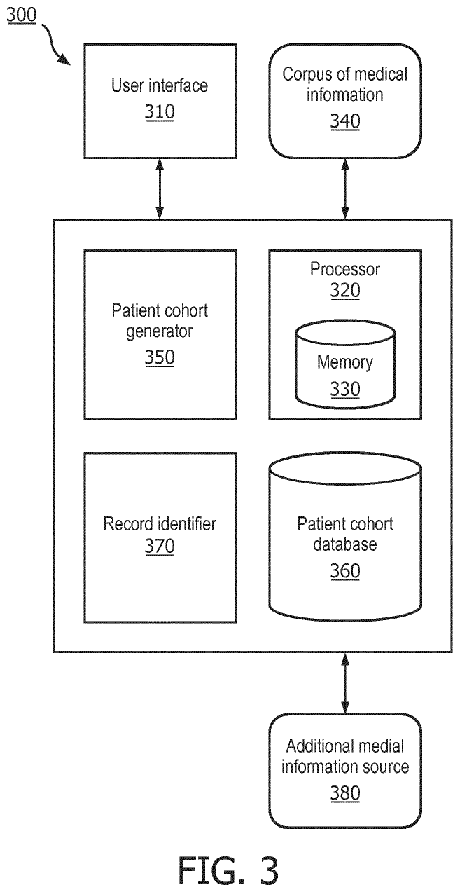

Referring to FIG. 3, in one embodiment, is a schematic representation of a medical records system 300. System 300 can comprise any ofthe modules, elements, databases, processors, and/or other components described or otherwise envisioned herein.

According to an embodiment, system 300 comprises a user interface 310 to receive a query from a user, to track user interaction with the system, and/or to provide identified information to the user. The user interface can be any device or system that allows information to be conveyed and/or received, such as a speaker or screen, among many other types of user interfaces. The information may also be conveyed to and/or received from a computing device or an automated system. The user interface may be located with one or more other components of the system, or may located remote from the system and in communication via a wired and/or wireless communications network.

According to an embodiment, system 300 comprises a processor 320 which performs one or more steps of the method, and may comprise one or more of the modules. Processor 320 may be formed of one or multiple modules, and can comprise, for example, a memory 330. Processor 320 may take any suitable form, including but not limited to a microcontroller, multiple microcontrollers, circuitry, a single processor, or plural processors. Memory 330 can take any suitable form, including a non-volatile memory and/or RAM. The non-volatile memory may include read only memory (ROM), a hard disk drive (HDD), or a solid state drive (SSD). The memory can store, among other things, an operating system. The RAM is used by the processor for the temporary storage of data. According to an embodiment, an operating system may contain code which, when executed by the processor, controls operation of one or more components of system 300.

According to an embodiment, system 300 comprises a patient cohort generator 350, which may be a processor, a component of one or more processors, and/or a software algorithm. The patient cohort generator 350 creates one or more patient cohorts as described or otherwise envisioned herein. The patient cohort will comprise a plurality of patients that are related based on one or more parameters. For example, patients may be related based on a clinical context, such as illness, symptoms, treatment, medical history, and/or other clinical contexts. Patients may be related based on patient demographics such as sex, age, background, and/or other patient demographics. The patients may be related based on which record or records a user most commonly accesses or reviews for a patient. Patients may be identified as being related based on a combination of several of these and/or other parameters. The generated patient cohort will further comprise an identification of one or more types of information, such as medical records, that are most commonly accessed, reviewed, or otherwise utilized by users of the medical record system in regard to the patients in the patient cohort.

According to an embodiment, patient cohort generator 350 creates one or more patient cohorts by tracking the activity of one or more users of the system, identifying patient information accessed by the one or more users through the electronic medical record interface, identifying one or more patient parameters associated with the patient in order to create the patient cohort of related patients, and associating two or more patients into a patient cohort based on the one or more patient parameters. The patient cohort generator 350 also identifies one or more types of information most commonly accessed by the users for the particular patient cohort, and associates the identified one or more types of information or records with the patient cohort. The patient cohort generator 350 can further identify portions of snippets of these records to preferentially display. The patient cohort generator 350 may consult additional information to modify the patient cohort and/or the identified one or more types of information or records associated with the patient cohort. For example, the patient cohort generator 350 may consult a source 380 of additional medical information such as Medscape, PubMed, Wikipedia, medical journals, other knowledge-based databases, clinician-curated data, and many more sources.

According to an embodiment, patient cohort generator 350 creates one or more patient cohorts using a corpus of medical information 340, such as information about a plurality ofpatients. The corpus of medical information may be a database of patient information associated with data regarding records which are commonly accessed for those patients.

According to an embodiment, the patient cohort generator 350 stores the generated patient cohort and associated record types or information in a database 360, which may be a component of the system or may be stored locally or remotely and in periodic and/or continuous communication with the system.

According to an embodiment, system 300 comprises a record identifier 370, which may be a processor, a component of one or more processors, and/or a software algorithm. Record identifier 370 receives, analyzes, and/or interprets a request for information about a patient received via user interface 310. The request can be from a clinician or any other user of the medical records system, including a patient. The information about the patient can be medical history, patient parameters, and/or medical records, among many other types of information.

Record identifier 370 associates the patient with one of the generated plurality of patient cohorts, based at least in part on one or more patient parameters. The patient may be associated with a patient cohort for which the patient is most similar, where similarity may be based on, for example, a threshold, a number of similar or dissimilar parameters, severity or range of one or more of the parameters, input from a programmer or user of the system, demographics, record types, illness, and/or many other patient factors.

Record identifier 370 may analyze additional information about the patient, such as patient's notes, social media, activity patterns, and/or other data sources, in order to facilitate identification of the proper patient cohort, and/or to reduce redundancy in the system.

Record identifier 370 identifies, based on the patient cohort with which the patient is associated, the one or more types of information or records associated with that cohort and most commonly accessed or utilized. Record identifier 370 may also identify, highlight, or otherwise provide specific portions or snippets of the identified records. The record identifier 370 may then send the patient information, comprising identified records and/or identified portions or snippets of records, to the user interface 310, a server or database, or to another location.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A system configured to analyze electronic medical records, the system comprising:
a user interface configured to receive input from a user as the user reviews one or more electronic medical records, and further configured to receive a request for patient information; and
a processor comprising:
a patient cohort generator configured to: (i) track the user input; (ii) identify, based on the user input, patient information accessed through the user interface, and further identify one or more patient parameters associated with the patient; (iii) associate two or more patients into a patient cohort based on the one or more patient parameters; (iv) identify, for the patient cohort, one or more types of information most commonly accessed by the users; and (v) associate the identified one or more types of information with the patient cohort; and
a record identifier configured to: (i) associate the patient for whom patient information is requested with a patient cohort; and (ii) identify, based on the patient cohort with whom the patient is associated, the one or more types of information associated with that cohort.

2. The system of claim 1, further comprising a patient cohort database configured to store information about one or more generated patient cohorts.

3. The system of claim 1, wherein the patient cohort generator is further configured to identify a specific user of the user interface among a plurality of users, and further configured to track the user input for the identified specific user.

4. The system of claim 3, wherein the identification of the specific user of the user interface is based at least on part on one or more favorable outcomes for one or more patients.

5. The system of claim 1, wherein the patient cohort generator is further configured to refine the associated identified one or more types of information using data from an additional medical information source.

6. The system of claim 1, wherein the patient cohort identification by the record identifier is based at least in part on additional obtained information about the patient.

7. The system of claim 1, wherein the user interface is further configured to display one or more of the identified types of information associated with the identified patient cohort.

8. The system of claim 7, wherein the user interface is further configured to display a portion of the identified types of information associated with the identified patient cohort.

9. The system of claim 1, wherein:
the processor is further configured to determine whether the user is to be categorized as an expert user, and
in identifying one or more types of information most commonly accessed by the users, the processor is configured to identify one or more types of information most commonly accessed by expert users of the system.

10. The system of claim 9, wherein, in determining whether the user is to be categorized as an expert user, the processor is configured to analyze the user's input to determine an ease of use metric associated with the user, wherein users that display relatively higher ease of use of the system are categorized as expert users.

11. A method for analyzing electronic medical records, the method comprising the steps of:
generating a patient cohort, comprising the steps of:
tracking the activity of one or more users of an electronic medical record interface;
identifying, based on an analysis of the tracked activity, patient information accessed by the one or more users through the electronic medical record interface, and further identifying one or more patient parameters associated with the patient;
associating two or more patients into a patient cohort based on the one or more patient parameters;
identifying, for the patient cohort, one or more types of information most commonly accessed by the users; and
associating the identified one or more types of information with the patient cohort;
receiving, from a user, a request for information about a patient;
associating the patient, based on one or more parameters of the patient, with a patient cohort; and
identifying based on the associated patient cohort, the one or more types of information associated with that cohort.

12. The method of claim 11, wherein the step of generating a patient cohort further comprises identifying a specific user of the electronic medical record interface for tracking.

13. The method of claim 12, wherein the identification of the specific user of the user interface is based at least on part on one or more favorable outcomes for one or more patients.

14. The method of claim 11, further comprising the step of refining the identified one or more types of information associated with the patient cohort using additional medical information.

15. The method of claim 11, further comprising the step of analyzing additional obtained medical information about the patient.

16. The method of claim 11, further comprising the step of displaying, on a user interface, the one or more types of information associated with the identified cohort.

17. The method of claim 16, wherein display the one or more types of information associated with the identified cohort comprises displaying only an identified portion of the one or more types of information.

18. The method of claim 11 further comprising:
determining whether the user is to be categorized as an expert user,
wherein identifying one or more types of information most commonly accessed by the users comprises identifying one or more types of information most commonly accessed by expert users of the system.

19. The system of claim 18, wherein determining whether the user is to be categorized as an expert user comprises analyzing the user's input to determine an ease of use metric associated with the user, wherein users that display relatively higher ease of use of the system are categorized as expert users.

20. A non-transitory machine-readable storage medium encoded with instructions for displaying portions of medical records, the non-transitory machine-readable storage medium comprising:
instructions for presenting a user interface to a plurality of users for reviewing one or more electronic medical records associated with a plurality of patients;
instructions for tracking use of the user interface by the plurality of users;
instructions for classifying at least one of plurality of users as an expert user based on tracked use for the expert user;
identifying a type of information commonly accessed by the expert user for patients of a first cohort;
receiving, via the user interface, a request for data relating to a patient;
determining that the patient belongs to the first cohort;
locating at least one record stored for the patient that includes information of the type of information commonly accessed by the expert user for patients of a first cohort;
displaying at least one of a portion of the record, an identifier of the record, or a link to the record in response to the request for data relating to the patient.
